(19) United States
(12) Patent Application Publication
(10) Pub. No.: US 2002/0072053 A1
(43) Pub. Date: Jun. 13, 2002

(54) Immunoassay based on DNA replication using labeled primer

(76) Inventors: Alan J. McNally, Carmel, IN (US); Robert S. Wu, West Orange, NJ (US); Zhuyin Li, Flemington, NJ (US)

Correspondence Address: Marilyn L. Amick, Roche Diagnostics Corporation, 9115 Hague Road, Bldg. D, P.O. Box 50457, Indianapolis, IN, 46250-0457, US

(21) Appl. No.: 09/733,565

(22) Filed: Dec. 8, 2000

(51) Int. Cl. C12Q 1/68

(52) U.S. Cl. 435/6

(57) ABSTRACT

An immunoassay method based upon inhibition of a DNA polymerase enzyme accomplished by linking a ligand of the analyte to a primer through a covalent bond. The interaction between the primer-bound ligand and a receptor specific for the ligand inhibits the DNA polymerase enzyme from generating double stranded DNA. The degree of inhibition of double stranded DNA synthesis is inversely proportional to the concentration of analyte in the sample. The analyte is determined by measuring the formation of double stranded DNA, e.g., by a fluorescence DNA intercalation technique.

### BACKGROUND OF THE INVENTION

This invention relates to an immunoassay method for determining an analyte in a sample. More specifically, it relates to a method based upon DNA synthesis using an analyte-labeled primer.

During the last several years, much effort has been focused on developing a highly sensitive, non-isotopic immunoassay for the detection of various analytes. The ultimate goal in this effort is to develop a highly sensitive, homogeneous assay that is totally automated. Currently, automated homogeneous immunoassays such as Roche OnLine®, Syva Emit®, Abbott TDx and Microgenics CEDIA® are commercially available. These assays are convenient to use because they require no separation and washing steps, and they are easily automated. However, they have detection limits of around 10⁻⁹ M in their clinical applications. In addition, several heterogeneous immunoassays have been described including Abbott fluorescence polarization, DuPont Immuno-PCR, SeaLite Sciences bioluminescent hybridization immunoassay, Roche Diagnostics ElecSys® electrochemiluminescence technology and microarray-based ligand binding systems. These assay methods reportedly can detect a 10⁻¹¹ M or lower concentration of analytes. All of these methods, however, are time-consuming and difficult to use because they require special instruments as well as separation and washing steps.

### SUMMARY OF THE INVENTION

The present invention comprises a novel immunoassay method based upon the inhibition of a DNA polymerase enzyme. This is accomplished by linking a ligand of the analyte to a primer through a covalent bond. The interaction between the primer-bound ligand and a receptor specific for the ligand inhibits the DNA polymerase enzyme from generating double stranded DNA. The degree of inhibition of double stranded DNA synthesis is inversely proportional to the concentration of analyte in the sample and provides the basis for a DNA-based competition immunoassay. The analyte is determined by measuring the formation of double stranded DNA, e.g., by using a fluorescence DNA intercalation technique.

The present invention also comprises a method wherein the inhibition of DNA polymerase enzyme is accomplished by linking a ligand of the analyte to the enzyme. The interaction between the enzyme-bound ligand and its antibody sterically prevents DNA polymerase from acting.

The present invention also comprises a method wherein the DNA polymerase enzyme is inhibited by the interaction of a primer-bound antibody, Fab or Fab′2 fragment and the analyte.

### BRIEF DESCRIPTION OF THE DRAWINGS

FIG. 1 is a synthetic scheme illustrating the synthesis of an LSD-modified universal primer from an amino dT C₆ modified universal primer and LSD-biphenyl-NHS derivative as described in Example 1.

FIG. 2 illustrates titration of reaction time. In each reaction well, there were 4×10⁻¹³ moles of the M13 template, 8×10⁻¹³ moles of the LSD-modified universal primer, 10 units of the polymerase enzyme. Reaction time varied. Error bars represent duplicates.

FIG. 3 illustrates titration of the polymerase enzyme concentration. In each reaction well, there were 4×10⁻¹³ moles of the M13 template, 8×10⁻¹³ moles of the LSD-modified universal primer, and reaction time was fixed at 10 minutes. The concentration of the polymerase enzyme varied. Error bars represent duplicates.

FIG. 4 illustrates titration of the concentration of the template. 8×10⁻¹³ moles of the LSD-modified universal primer and 2 units of the polymerase enzyme were used for each reaction. Reaction time was 10 minutes. The concentration of the M13 template varied.

FIG. 5 illustrates titration of the ratio between the template and the primer. 1×10⁻¹³ moles of the M13 template and 2 units of the polymerase enzyme were used per reaction. Reaction time was fixed at 30 minutes. The molar ratio between the template and the primer varied. Error bars represent duplicates.

FIG. 6 illustrates DNA replication with modified universal primers. In each reaction well, there were 1×10⁻¹³ moles of the M13 template, 2 units of the polymerase enzyme and either 2×10⁻¹³ moles of the amino dT C₆ modified universal primer, 2×10⁻¹³ moles of the LSD-modified universal primer, or no primer (negative control). Reaction time was 30 minutes. Error bars represent duplicates.

FIG. 7 illustrates inhibition of DNA replication by analyte-specific antibody. To each reaction well, 2×10⁻¹³ moles of the LSD-modified universal primer were incubated with protein A purified LSD IgG antibodies at various titers for 10 minutes, then 1×10⁻¹³ moles of the M13 template and 2 units of the polymerase were added. Reaction time was 30 minutes. Error bars represent duplicates.

FIG. 8 illustrates specificity of the inhibition of DNA replication by antibody. Combinations of modified universal primers and antibodies against LSD or morphine were used. Error bars represent duplicates.

FIG. 9A illustrates a competition assay for LSD under the most cost-effective conditions. FIG. 9B illustrates a competition assay for LSD under the best DNA replication conditions. Error bars represent duplicates.

FIG. 10 illustrates the effect of salt concentration on the replication of DNA by the polymerase enzyme. Error bars represent duplicates.

FIG. 11 illustrates the effect of heat stress of urine samples on the intensity of fluorescence signal. Error bars represent duplicates.

FIG. 12 is a representative curve for a biotin competition assay using the method of the present invention. Concentration of biotin is plotted on the X-axis and fluorescence is plotted on the Y-axis.

FIGS. 13A and 13B are graphs comparing IgG, F(ab′)2, Fab′ and Fab′-primer tested using Roche CEA enzyme immunoassay kit. The dilution factor is plotted on the X-axis and absorbance is plotted on the Y-axis.

FIG. 14 is a chart showing DNA synthesis using Fab′ modified universal primer.

### DETAILED DESCRIPTION OF THE INVENTION

The present invention comprises a highly sensitive homogeneous immunoassay based on DNA synthesis. In one embodiment, the immunoassay is based on DNA synthesis with an analyte-labeled or antibody-labeled primer. The method comprises a DNA-based competitive enzyme inhibition assay system which combines highly specific antibody-antigen interaction technology with highly specific primer-template-polymerase interaction technology.

In a preferred embodiment, the present invention comprises a method for determining an analyte in a sample suspected of containing said analyte, said method comprising the steps of (a) under conditions favorable for DNA replication, combining said sample with a single stranded DNA template capable of replication, a deoxyribonucleotide precursor, a DNA polymerase enzyme, a receptor capable of binding with said analyte, a compound capable of generating a detectable signal in the presence of double stranded DNA, and a primer, said primer linked to a ligand or analog of said analyte, (b) monitoring the generation of double stranded DNA by said enzyme by measuring the signal produced by said compound, and (c) correlating the production of said signal with the presence or amount of said analyte in said sample.

A small organic analyte, LSD, was used to demonstrate the concept. This hapten was chosen for its high sensitivity requirement. With this analyte, the cutoff limits of 500 pg/ml can easily be met using the most cost-effective parameters chosen to readily commercialize such an assay. In fact, using short incubation times with cost-effective raw materials, limits of detection as low as 10⁻¹¹ M can be achieved. When the best conditions for maximum sensitivity were chosen, e.g., using more polymerase enzyme, etc., limits of detection as low as 10⁻¹² M were demonstrated.

The present DNA-based immunoassay system may also be applied to the analysis of large molecular weight protein analytes such as carcinoembryonic antigen (CEA). In this case, the smallest functional fragment of the antibody, Fab, is linked to the primer through a similar covalent bond, but at a different location on the primer. Interaction between the protein analyte in the sample and its primer linked antibody inhibits the DNA polymerase enzyme.

An alternate method for inhibition of the DNA polymerase enzyme is realized by labeling the enzyme with an analyte near the enzyme's active site through random chemical modification or site-directed mutagenesis. However, when comparing the modification of the enzyme with the analyte versus labeling of the primer with the analyte, the latter would require less time and fewer materials to accomplish.

Several modifications may be made to enhance the marketability of the DNA-based competitive immunoassay of the present invention. For example, a more stable polymerase enzyme which can also withstand a high salt environment would be desirable, and several polymerases of this type have been suggested in the literature. Also desirable are specific DNase inhibitors which can inhibit undesirable DNase enzymes present in urine. The inhibitors, of course, should not alter the activity of the polymerase enzyme. Finally, the method for detecting the formation of double stranded DNA using a fluorescence method (PicoGreen®) may be modified by employing a UV-visible absorbance-based detection method.

The basic elements required by this invention are template, primer, deoxyribonucleotide precursor, DNA polymerase enzyme and antibody or receptor specific for analyte. The template is a single stranded DNA, either random or with a known sequence. The primer is a small piece of single stranded DNA with a free 3′—OH group, which dictates the starting position of DNA replication. The ideal primer is comprised of 5 to 30 base pairs, preferably 10 to 20 base pairs. The primer sequence should be complementary to the starting sequence of the template. The deoxyribonucleotides act as substrates for the enzyme reaction. DNA polymerase is an enzyme that catalyzes the step-by-step addition of deoxyribonucleotide residues to a DNA chain. Examples of polymerase enzymes that can be used in the present invention include various polymerases from different species of Thermus aquaticus. Finally, the receptor used in the method of the present invention is an antibody, monoclonal or polyclonal, or antibody fragment that is specific for the targeted analyte.

Analyte refers to the substance, or group of substances, whose presence or amount thereof in a sample is to be determined and is meant to include substances such as haptens, drugs and drug derivatives, hormones, protein antigens and oligonucleotides.

Antibody, or preferably, receptor, means a specific binding partner of the analyte and includes any substance, or group of substances, which has a specific binding affinity for the analyte ligand to the exclusion of other substances.

Ligand means any substance, or group of substances, which behaves essentially the same as the analyte with respect to binding affinity of the receptor for the analyte and is meant to include any hapten, drug or drug derivative, hormone, protein, polypeptide, or nucleotide.

Conditions favorable for DNA synthesis or replication are well known to those skilled in the art to which the present invention pertains and are exemplified in the specific embodiments described in the examples herein.

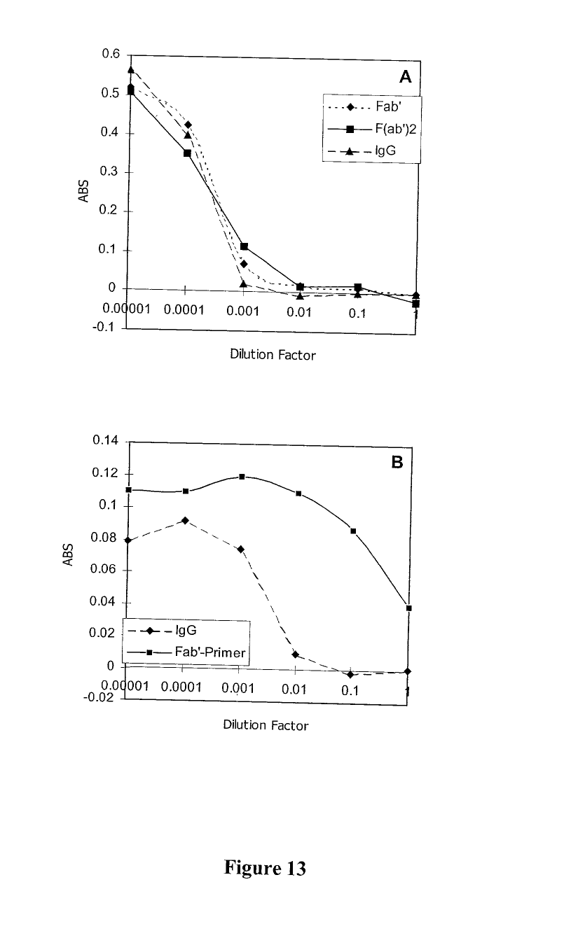

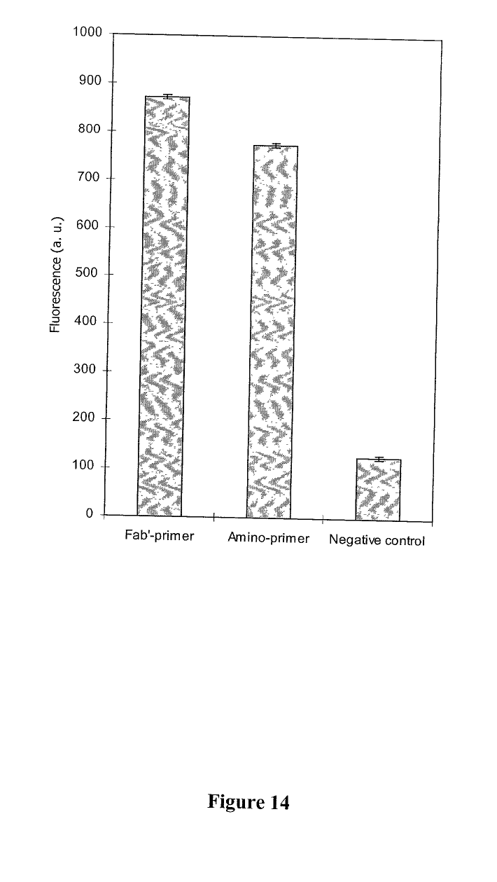

In a preferred embodiment of the present invention, which is based on competitive enzyme inhibition, the primer is labeled near it 3′ end with a ligand of choice through a covalent bond to, e.g., thymine. The ligand-labeled primer competes with analyte in the sample for the ligand-specific receptor. In the presence of free analyte, the receptor binds to the analyte in the sample in a competitive kinetic mode. Ligand-labeled primer is then able to anneal to the template, which in turn allows DNA polymerase to synthesize double stranded DNA (FIG. 13A). Conversely, in the absence of free analyte, the antibody binds to the primer-linked ligand, DNA replication is inhibited, and double stranded DNA is not synthesized. It is theorized that the inhibition of DNA replication is due to the spatial effect caused by the antibody-ligand complex, which may inhibit the annealing of the primer to the template (FIG. 13B) or inhibit the function of the polymerase enzyme (FIG. 13C).

Any detection method capable of distinguishing double stranded DNA may be used with the present invention. A number of methods such as UV absorption, chemical luminescence, fluorescence, fluorescence polarization, ³²P labeling, and gel electrophoresis have been used previously to distinguish double stranded DNA from single stranded DNA, and such methods will be familiar to those skilled in the art to which the present invention pertains. A preferred detection method that is especially suitable for automated high throughput analyzers is one using DNA intercalators. A DNA intercalator is a small molecule that fits into the grooves of double stranded DNA and binds noncovalently to the DNA. The absorbance or fluorescence intensity of DNA intercalators is different when binding non-specifically to a single stranded DNA or double stranded DNA. The fluorescence intercalator technique provides the desired high sensitivity, fast reaction kinetics, and ease of automation required for highly sensitive non-isotopic assays. Examples of fluorescent DNA intercalators include acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine, YOYO-1 and others. PicoGreen, a commercially available fluorescence DNA intercalator, was used in a preferred embodiment as a fluorescent probe for the DNA synthesis-based immunoassay of the present invention. The PicoGreen molecule readily intercalates into the grooves of the double stranded DNA, and its fluorescence quantum yield, or intensity, increases dramatically upon this intercalation, providing a good method for distinguishing double stranded DNA from single stranded DNA.

The assay of the present invention is suitable for detection of analytes of interest such as small organic drug haptens or large, clinically significant proteins. As used herein, the terms “analyte” and “analyte of interest” pertain to a substance which is to be detected and preferably quantified. Analytes may be inorganic or organic, though typically they are organic. Analytes may be naturally occurring or synthetic. Examples of classes of organic analytes include biological molecules such as amino acids, proteins, glycoproteins, lipoproteins, saccharides, polysaccharides, lipopolysaccharides, fatty acids and nucleic acids. Examples of organic analytes include antibodies, antigens, haptens, enzymes, hormones, steroids, vitamins, oligonucleotides and pharmacological agents.

In a preferred embodiment, (+)-lysergic acid diethylamide (LSD) was used as the analyte of interest. The detection of LSD in body fluids is difficult because the quantities typically ingested are very small (100-250 μg), and LSD is rapidly and extensively metabolized in vivo. Therefore, the development of a highly sensitive assay for LSD is challenging. Using the method described herein, the present DNA synthesis-based immunoassay detects LSD and other analytes with limits of detection from 10⁻⁸ to as low as 10⁻¹² M (5×10⁻¹⁸ moles).

It has been found for purposes of the present invention that the best conditions for the highest level of double stranded DNA formation comprise high enzyme, template, and primer concentrations, along with longer reaction times. However, considering assay costs and assay timing, preferred conditions comprise a 30-minute reaction time, 1×10⁻¹³ moles (250 ng/ml) M13 template per reaction, 2 units of polymerase enzyme per reaction, and a template to primer molar ratio of 1:2. A reaction of ten minutes is less preferred because limited signal was observed under the sensitivity requirement of an LSD assay. In addition, a reaction time of 60 minutes is less preferred because it would seriously affect the first reported result and total throughput of the automated analyzer. There is great flexibility in the assay method of the present invention, and specific assay conditions will largely depend on costs, convenience, materials available, time, and conditions of enzyme, template and primer.

Another aspect of the present invention relates to a test kit useful for conveniently performing the assay method of the invention for the determination of an analyte. To enhance the versatility of the subject invention, reagents useful in the method of the invention can be provided in packaged combination, in the same or separate containers, in liquid or lyophilized form so that the ratio of the reagents provides for substantial optimization of the method and assay. The reagents may each be in separate containers, or various reagents can be combined in one or more containers depending on the cross-reactivity and stability of the reagents.

A test kit of the present invention comprises, in packaged combination, a single stranded DNA template capable of replication, a primer linked to a ligand or analog of the analyte, the primer comprising a sequence complementary to the starting sequence of the primer, a DNA polymerase enzyme, a receptor capable of binding with the analyte, and a compound capable of generating a detectable signal in the presence of double stranded DNA.

Alternatively, the test kit may comprise a primer linked to a receptor capable of binding with the analyte, a template comprising the primer sequence and capable of replication, a DNA polymerase enzyme, and a compound capable of generating a detectable signal in the presence of double stranded DNA.

In the following examples, DNA polymerase (Klenow fragment) was purchased from Amersham Life Science (Arlington Heights, Ill.). M13 mp18 (+) single stranded DNA template (˜7400 bases) from E. coli was obtained from Pharmacia Biotech (Piscataway, N.J.). The amino dT C₆ modified M13 universal primer was synthesized by Oligos (Wilsonville, Oreg.). PicoGreen was purchased from Molecular Probes (Eugene, Oreg.). Deoxyribonucleotides (dNTP) were manufactured by Roche Molecular Systems (Branchburg, N.J.). LSD-NHS derivatives were synthesized by the Roche Diagnostic Systems as described in European patent application EP 816,364. LSD polyclonal antibodies were produced by Roche Diagnostic Systems (Branchburg, N.J.). All other chemicals were obtained from Fisher or Sigma. All solvents used for HPLC analysis were HPLC grade.

### EXAMPLE 1

### Synthesis of Primer-LSD Conjugate

A modified universal primer for the M13 template was used. The sequence for this modified primer was 5′ GTA AAA CGA CGG CCX GT 3′ where X is an amino dT C₆ linker (1).

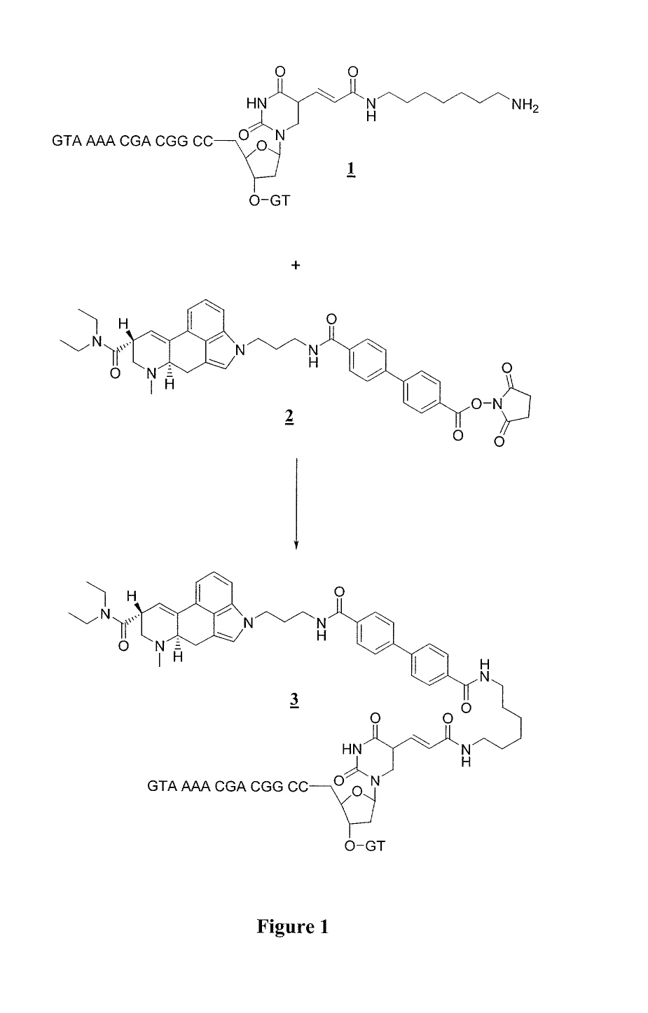

The amino dT C₆ modified universal primer for the single stranded M13 template was dissolved in 80 mM sodium phosphate, pH 8 buffer at a concentration of 1.1 μg/μl. 70 μl of 100% DMF was added dropwise to 70 μl of the amino dT C₆ modified universal primer stock solution with gentle vortexing. The LSD-NHS derivative (2) was dissolved in 100% DMF at a concentration of 10 mg/ml, and 30 ml of the LSD derivative stock solution was added dropwise to the amino dT C₆ modified universal primer in 50% DMF/sodium phosphate buffer solution with gentle vortexing. The reaction was carried out overnight at room temperature in a glass container protected from light. The final concentration of the amino dT C₆ modified universal primer was 8.0×10⁻⁵ M, and the final concentration of the LSD derivative was 4×10⁻³ M; the concentration of DMF was 59%. See FIG. 1 for a schematic representation of the synthesis.

### EXAMPLE 2

### Preparation of Primer-LSD Conjugate

The purification of the LSD-modified universal primer (3) was carried out using high pressure liquid chromatography (HPLC). The HPLC system consisted of a Waters 600 multisolvent delivery system, a Waters 490 multiwavelength detector and a Waters 420 fluorescence detector (Waters, Milford, Mass.). Separation was accomplished using a reverse phase μBondapak C₁₈ column (Waters, Milford, Mass.) and a mobile phase of 0.1 M triethylamine acetate (pH 6.5) with increasing acetonitrile concentrations (10-20%) at a flow rate of 2 ml/minute. The eluent was monitored at 260 nm for UV absorbance, and fluorescence from the LSD derivative was also monitored with an excitation wavelength of 340 nm and an emission wavelength of 425 nm. The LSD-modified universal primer peak fraction was collected and dialyzed in dialysis tubing having a molecular weight cutoff of 3 kDa, step down dialysis from 50% CH₃CN/10 mM sodium phosphate, pH 7.5 at 25% CH₃CN intervals, 252 fold dialysis at each CH₃CN concentration except 25³ fold dialysis in 0% CH₃CN buffer. Finally, the LSD-modified universal primer was lyophilized and kept at −20° C. until use. The yield (20-30%) of the LSD-modified universal primer obtained was estimated by the reduction of the absorbance peak at 260 nm as compared to the absorbance of the original amino dT C₆ modified primer.

Reaction conditions were established in which both the LSD derivative and the amino dT C₆ modified universal primer were soluble. In addition, reaction conditions were developed in which nucleophilic chemical reactions between amino groups of the primer and the NHS group of the LSD derivative were favored. The concentration of organic solvent, the solution pH and the material of the container were key factors in determining the yield for the formation of the LSD-modified universal primer. Under the present conditions, about 20% of amino dT C₆ modified universal primer is converted into LSD-modified universal primer.

Under the HPLC purification conditions, the singular components of the reaction mixture were well separated. Amino dT C₆ modified universal primer was eluted at 8 minutes, followed by the LSD-modified universal primer at 23 minutes. The hydrolyzed LSD derivative was eluted at 26 minutes, and the LSD derivative was eluted at 41 minutes. Under these conditions, the amino group is not protonated. An unprotonated amino group is expected to react faster to NHS than a protonated one.

### EXAMPLE 3

### Conditions for DNA Synthesis

A series of titrations was conducted in order to obtain optimal DNA synthesis conditions. Amino dT C₆ modified universal primer was used for these titrations.

### EXAMPLE 3(a)

### Reaction Time

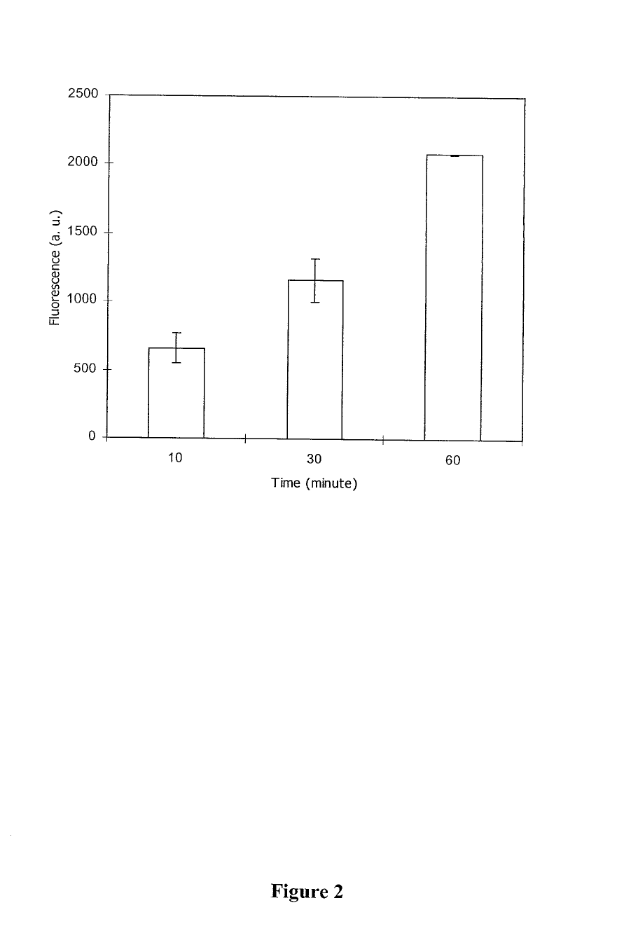

In order to determine the time required for the assay system to produce detectable signals, reagents were added into a 96-well microtiter fluorescence plate (Lab System) in the following order: 10 μl 10 mM Tris buffer, pH 7.5, 1.2 μl solution containing 4×10⁻¹³ moles of the template, 5 μl of primer solution containing 8×10⁻¹³ moles of the amino dT C₆ modified universal primer, 1 μl of enzyme solution containing 10 units of polymerase enzyme, 8 μl of dNTP at 10 mM concentration. All reagents were diluted in 10 mM Tris buffer, pH 7.5, except for the primer which was diluted in 500 mM Tris buffer, pH 7.5, 0.1 M MgCl₂, 10 mM dithiothreitol (DTT) and 0.5 mg/ml bovine serum albumin (BSA) (primer buffer). After incubation at 37° C. for 10, 30 or 60 minutes, 4.8 μl of 0.2 M EDTA was added to the well to stop the reaction, followed by 250 μl of PicoGreen reagent, at 1:200 dilution from the manufacturer's stock solution, to detect the concentration of double stranded DNA formation. Measurement of fluorescence from the double stranded DNA intercalated PicoGreen was carried out using a Fluoroscan II system (Lab System, Inc.). The results are shown in FIG. 2. Each data point represents the mean of duplicates. The results demonstrate that the longer the reaction time, the more double stranded DNA was formed.

### EXAMPLE 3(b)

### Polymerase Enzyme Concentration

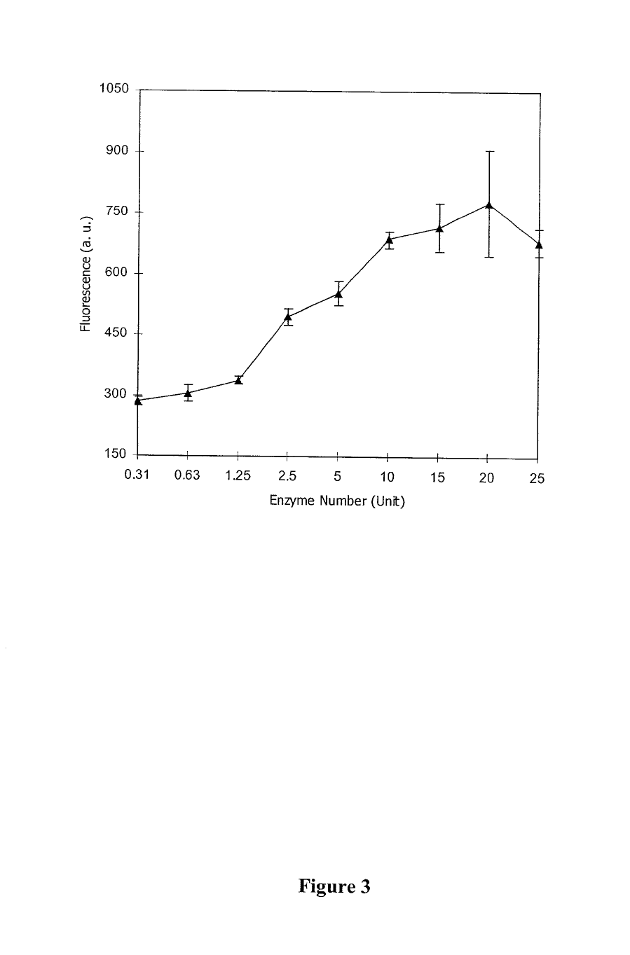

To determine the optimal polymerase enzyme concentrations for DNA synthesis, reagents were added into a 96-well microtiter fluorescence plate (Lab System) in the following order: 10 μl 10 mM Tris buffer, pH 7.5, 1.2 μl solution containing 4×10⁻¹³ moles of the template, 5 μl of primer solution containing 8×10⁻¹³ moles of the amino dT C₆ modified universal primer, 1 μl of enzyme solution containing 0.31, 0.63, 1.25, 2.5, 5, 10, 15, 20 and 25 units of polymerase enzyme, and 8 μl of dNTP at 10 mM concentration. All reagents were diluted in 10 mM Tris buffer, pH 7.5, except for the primer, which was diluted in 500 mM Tris buffer, pH 7.5, 0.1 M MgCl₂, 10 mM DTT and 0.5 mg/ml BSA (primer buffer). After incubation at 37° C. for 10 minutes, 4.8 μl of 0.2 M EDTA was added to the well to stop the reaction, followed by 250 μl of PicoGreen reagent at 1:200 dilution. Fluorescence from the double stranded DNA intercalated PicoGreen was measured using a Fluoroscan II system (Lab System, Inc.). The results are shown in FIG. 3. Each data point represents the mean of duplicates.

The final concentration of double stranded DNA increased as polymerase concentrations increased and then leveled off. The change in the reaction times affected the final concentration of the double stranded DNA; however, it did not change the trend of the reaction direction.

### EXAMPLE 3(c)

### Template Concentration

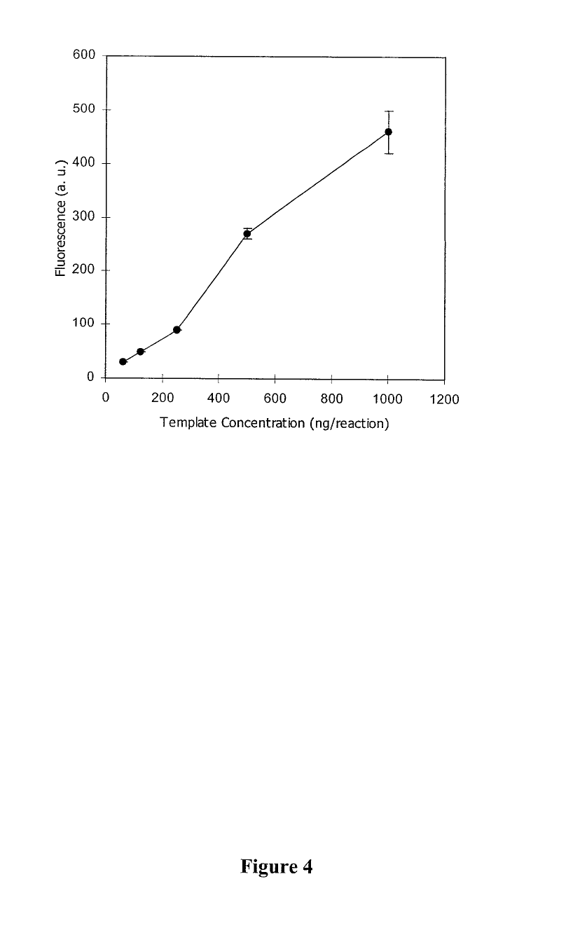

In order to determine the optimal template concentration for DNA synthesis, reagents were added into a 96-well microtiter fluorescence plate (Lab System) in the following order: 10 μl 10 mM Tris buffer, pH 7.5, 1.2 μl solution containing from 0.24×10⁻¹³ to 4×10⁻¹³ moles of the template, 5 μl of primer solution containing 8×10⁻¹³ moles of the amino dT C₆ modified universal primer, 1 μl of enzyme solution containing 2 units of polymerase enzyme, 8 μl of dNTP at 10 mM concentration. All reagents were diluted in 10 mM Tris buffer, pH 7.5, except for the primer which was diluted in 500 mM Tris buffer, pH 7.5, 0.1 M MgCl₂, 10 mM DTT and 0.5 mg/ml BSA (primer buffer). After incubation at 37° C. for 10 minutes, 4.8 μl of 0.2 M EDTA was added to the well to stop the reaction, followed by 250 μl of PicoGreen reagent at 1:200 dilution. Fluorescence was measured using a Fluoroscan II system. The results are shown in FIG. 4.

The Kₘ for the DNA polymerase enzyme, in terms of primer-template complex, was 5 nM. In this example, all template concentrations tested were below the Kₘ of the polymerase enzyme (more template is expected to decrease the sensitivity and would not be cost-effective), thus resulting in more primer-template complex formation when more template was used. This resulted in more double stranded DNA synthesized with high concentration of primer-template complex).

### Example 3(d)

### Template/Primer Ratio

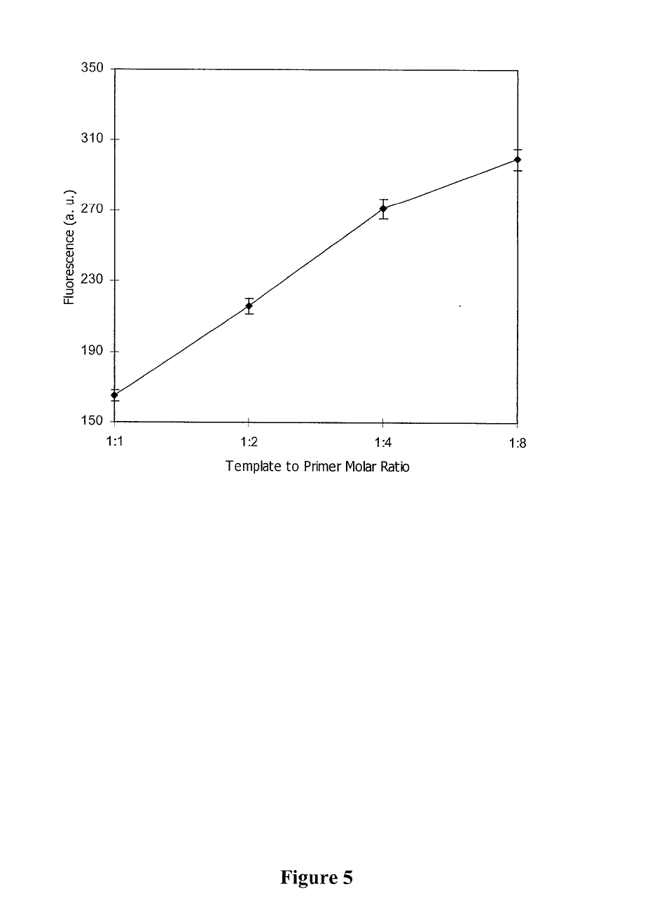

In order to determine the optimal ratio between template required for the assay system and primer for double stranded DNA formation, reagents were added into a 96-well microtiter fluorescence plate (Lab System) in the following order: 10 μl 10 mM Tris buffer, pH 7.5, 1.2 μl solution containing 1×10⁻¹³ moles of the template, 5 μl of primer solution containing from 1×10⁻¹³ to 8×10⁻¹³ moles of the amino dT C₆ modified universal primer, providing a ratio between template and primer of from 1:1 to 1:8, 1 μl enzyme solution containing 2 units of polymerase enzyme, and 8 μl of dNTP at 10 mM concentration. All reagents were diluted in 10 mM Tris buffer, pH 7.5, except for the primer, which was diluted in 500 mM Tris buffer, pH 7.5, 0.1 M MgCl₂, 10 mM DTT and 0.5 mg/ml BSA (primer buffer). After incubation at 37° C. for 30 minutes, 4.8 μl of 0.2 M EDTA was added to the well to stop the reaction, followed by 250 μl of PicoGreen reagent at 1:200 dilution. Fluorescence was measured on the Fluoroscan II system. The results are shown in FIG. 5.

When the template concentration was fixed at 1×10⁻¹³ moles per reaction (4.2 nM final reaction concentration), and the template to the primer molar ratio varied from 1:1 to 1:8, the double stranded DNA formation increased as the primer concentration increased.

### EXAMPLE 4

### DNA Synthesis with LSD Labeled Primer

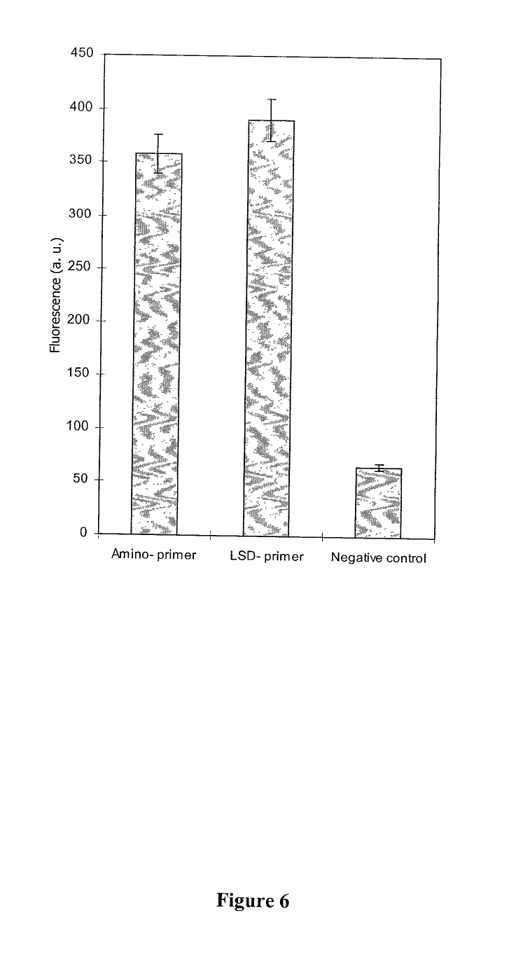

To determine whether binding an LSD derivative to different primers has an effect on DNA synthesis, the amino dT C₆ modified universal primer and the LSD-modified universal primer were tested and compared in DNA synthesis experiments. In each reaction well, the final concentration of both of the primers was 8.2 nM, the template concentration was 4.1 nM or 10 μg/ml, enzyme concentration was 2 units per reaction well, and dNTP concentration was 0.3 mM. The final buffer concentration was 108 mM, and the incubation time was 30 minutes. The results are shown in FIG. 6.

The degree of DNA synthesis was not changed when the LSD-modified universal primer was used instead of amino dT C₆ modified primer. Under selected reaction conditions, a signal to noise (negative control) ratio of about 5:1 was obtained for both modified universal primers. Noise is defined as the fluorescence signal produced from the DNA synthesis reaction without the use of any primer. With this example, it was demonstrated that the spatial effect induced by linking the LSD derivative (702 Da molecular weight) to the primer had no significant effect in causing detectable differences in DNA synthesis.

### EXAMPLE 5

### Inhibition of DNA Synthesis by Anti-LSD Specific Antibody

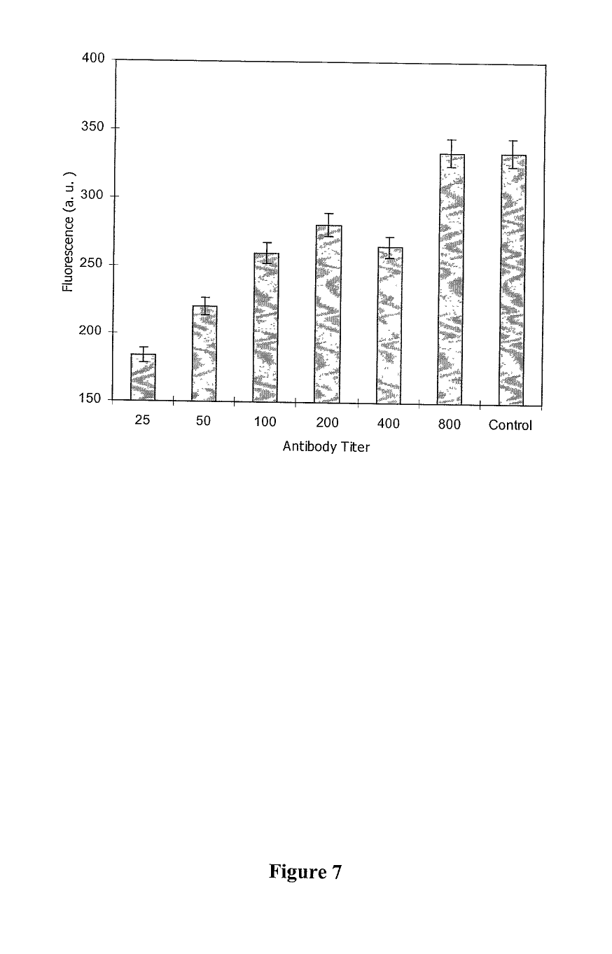

In a 96-well microtiter fluorescence plate, 5 μl 10 mM Tris buffer and 2×10⁻¹³ moles of LSD-modified universal primer in 5 μl of primer buffer were incubated for 10 minutes with 5 μl of anti-LSD antibody at titers of 1:25, 1:50, 1:100, 1:200, 1:400 and 1:800. The polyclonal antibodies used were generated using an immunogen comprising an LSD analog derivatized through the indole nitrogen and conjugated to bovine thyroglobulin (BTG) prepared as described in European patent application EP 816,364. Antibodies were diluted in 10 mM Tris buffer, pH 7.5. Both anti-LSD serum and protein A purified LSD IgG antibody from the serum were tested. After 10 minutes incubation, 1×10⁻¹³ moles of M13 template and 2 units of polymerase enzyme were added into the well and allowed to react 30 minutes. The results are shown in FIG. 7; error bars represent duplicates.

Anti-LSD serum and LSD IgG antibody purified from the serum by protein A immunoaffinity chromatography were used to inhibit DNA synthesis with the LSD-modified universal primer. Once anti-LSD antibody bound to the primer-linked LSD, DNA synthesis was inhibited. The degree of DNA synthesis inhibition was proportional to the concentration of antibodies. However, at very high antibody concentrations (low titer), an increase in fluorescence intensity was observed, possibly due to the non-specific interaction between protein and PicoGreen. The mechanism of inhibition may be due to the spatial effect caused by the antibody binding preventing the primer and the template from annealing, as illustrated in FIG. 13B; or the primer and template annealed, but the polymerase enzyme could not bind to the primer in an appropriate manner for catalysis, as shown in FIG. 13C.

### EXAMPLE 6

### Specificity of Inhibition of DNA Replication by Antibody

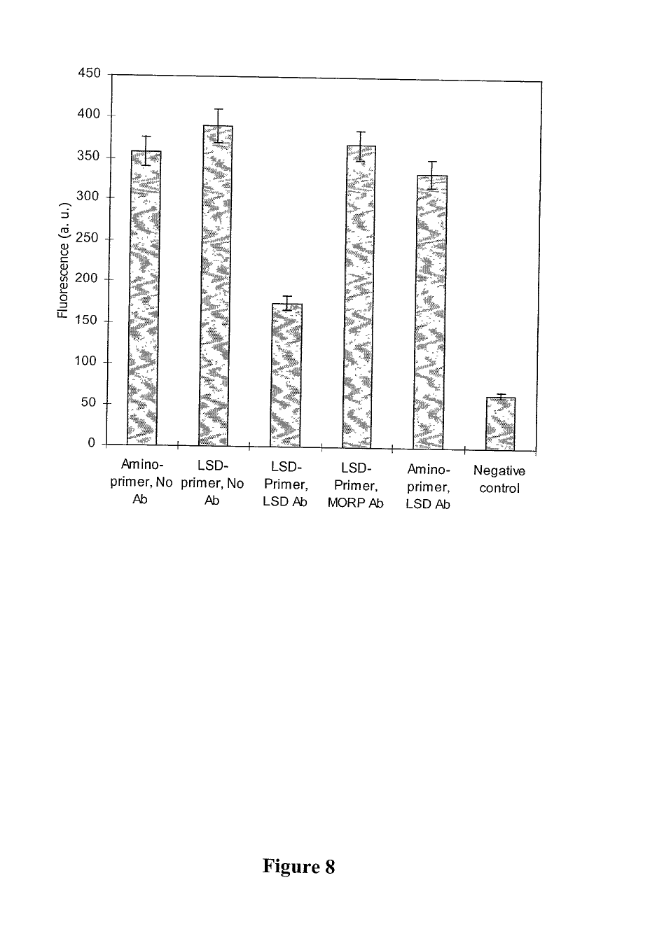

The specificity of antibody-caused inhibition was verified using combinations of modified universal primers and antibodies generated against LSD and morphine. In a 96-well microtiter fluorescence plate, 5 μl of primer solution containing 2×10⁻¹³ moles of the amino dT C₆ modified universal primer or the LSD-modified universal primer were incubated with 10 μl of Tris buffer, pH 7.5 or 10 μl of protein A purified LSD IgG antibody or 10 μl morphine antibody. Both antibodies were diluted in Tris buffer, pH 7.5 at 1:50 titer. The mixture was incubated for 10 minutes at room temperature, then 1×10⁻¹³ moles of template, 2 units of enzyme and 1 μl dNTP were added to the well, and fluorescence signal measurements were made. The results are shown in FIG. 8. Error bars represent duplicates.

The results demonstrate that morphine antibody used at similar total protein concentrations as that of the LSD antibody did not cause inhibition and that LSD antibody did not inhibit DNA replication with the amino dT C₆ modified primer which was not labeled with LSD derivative. Therefore, the inhibition of DNA synthesis by analyte-specific antibody (LSD) was specific for the antibody-antigen interaction between LSD and its antibody.

### EXAMPLE 7

### Detection of Analyte in Buffer

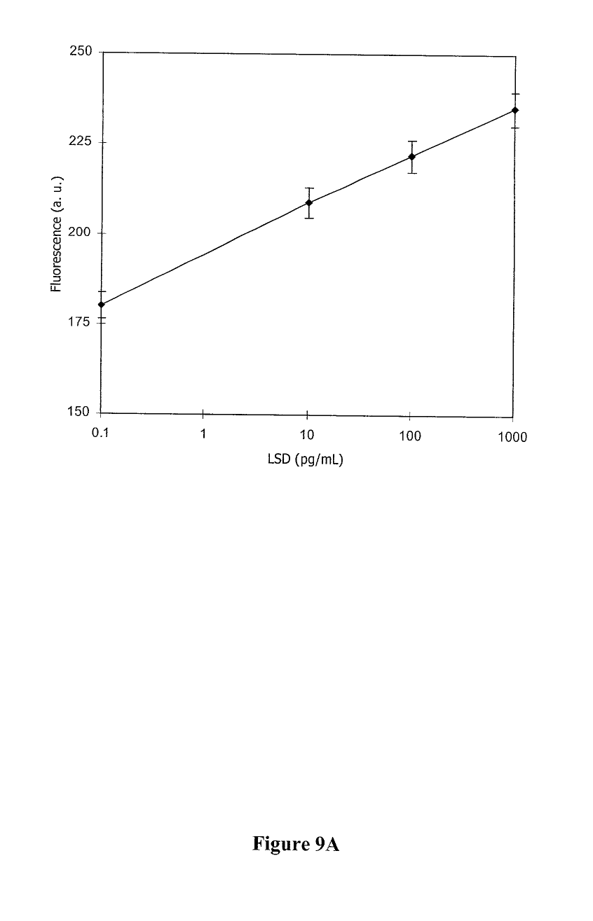

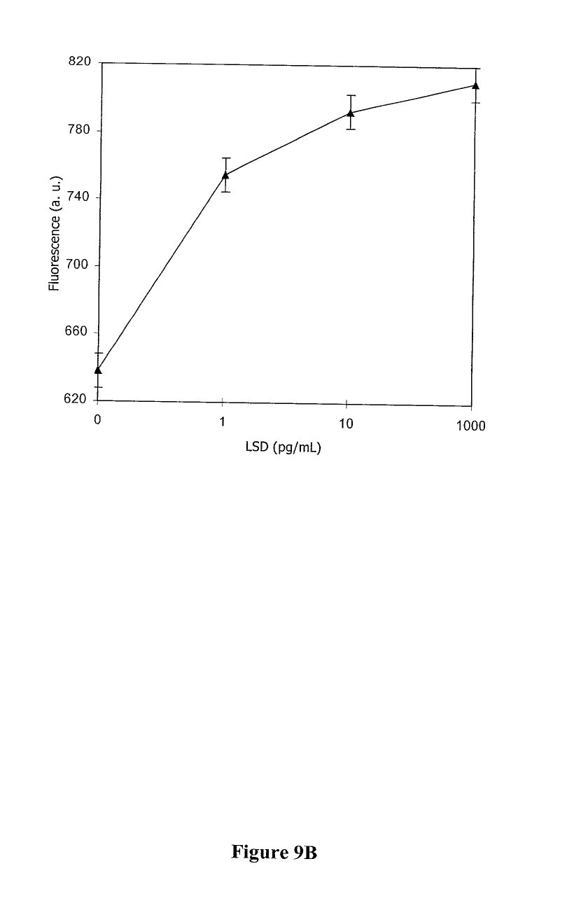

LSD was dissolved in DMSO at a concentration of 1 mg/ml, then further diluted to various concentrations in Tris buffer, pH 7.5. In each well of a 96-well microtiter fluorescence plate, 5 μl of the diluted LSD solution, for final concentrations of 0.1, 10, 100 and 100 pg/ml, were incubated with 5 μl of LSD antibody at a selected titer of 1:50 for 5 minutes, then 5 μl of the LSD-modified universal primer was added, and the mixture was incubated for another 5 minutes. These incubations between antibody and analyte were conducted to ensure the interaction between LSD and its antibody. Other reagents for DNA synthesis, as previously described, were added to each well subsequently, and the reaction time was 30 or 60 minutes. The fluorescence signal was detected as described above. Results are shown in FIGS. 9A and 9B. Error bars represent duplicates.

A competition system with free LSD to prevent the antibody inhibition of DNA synthesis with LSD-modified universal primer was also investigated. Two experiments were carried out. In the first, the most cost-effective parameters were used (FIG. 9A), and the second experiment was set up to determine the ultimate limit of detection for this type of competitive assay system (FIG. 9B). The lowest antibody concentration at which DNA synthesis was completely inhibited with the LSD antibodies was used in both experiments. Free LSD in the buffer competed with the primer-linked LSD to bind the antibody, allowing proportional amounts of the primer to anneal to the template. This resulted in proportional levels of double stranded DNA formation. A limit of detection for LSD was estimated to be 4 pg/ml, or approximately 10⁻¹¹ M, when the most cost-effective method was used. Without considering cost, the lowest limit of detection for LSD that could be reached was 10⁻¹² M (5×10⁻¹⁸ moles). Thus it was demonstrated that a competition binding system can be set up for the detection of LSD, and that sensitivities as low as 10⁻¹² M for analyte concentration can be obtained.

### EXAMPLE 8

### Detection of Analyte in Sample in the Presence of Salt

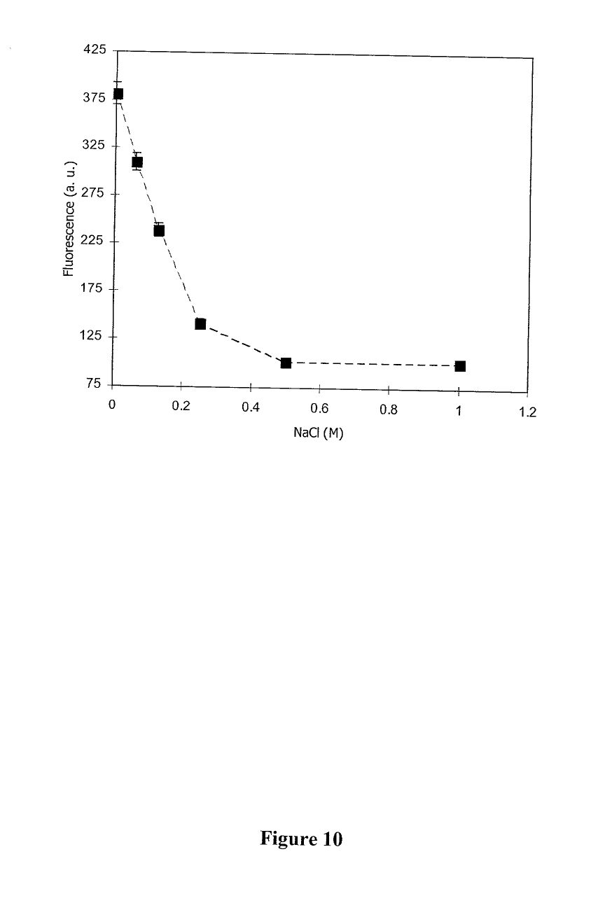

5 μl of 250 ng/ml LSD in Tris buffer, pH 7.5, containing various concentrations of NaCl and 5 μl of LSD antibody at a selected titer were mixed in each reaction well and incubated for 5 minutes at room temperature. Then 5 μl of the LSD-modified universal primer was added, and the solution was allowed to incubate for another 5 minutes. Incubation was conducted to allow reactions between LSD and its antibody. Subsequently, other reagents were added and the formation of double stranded DNA was detected by the fluorescence method described above. Results are shown in FIG. 10. Error bars represent duplicates.

The present invention is useful for detecting analytes in body fluids such as blood, plasma, cerebrospinal fluid, urine etc. One consideration in applying this method to urine is the effect of salts in urine on the function of the DNA polymerase enzyme. The effect of salt concentrations on the function of the polymerase enzyme has been reported. FIG. 10 demonstrates that when salt (NaCl) concentrations were increased from 0 to 0.1 M, the production of double stranded DNA was suppressed by 50%. However, we believe that this problem can be overcome by selecting a polymerase enzyme which can withstand high salt concentrations.

### EXAMPLE 9

### DNA Synthesis with LSD Labeled Primer in Urine Samples

Ten fresh human urine samples were collected. Samples were used untreated or were heat stressed at 100° C. for 10 minutes before using. In each reaction well, 5 μl of heat stressed or untreated urine and 5 μl of 10 mM Tris buffer, pH 7.5, were used instead of 10 μl of Tris buffer (see Example 4), and other reagents were subsequently added. The formation of double stranded DNA was detected by PicoGreen fluorescence.

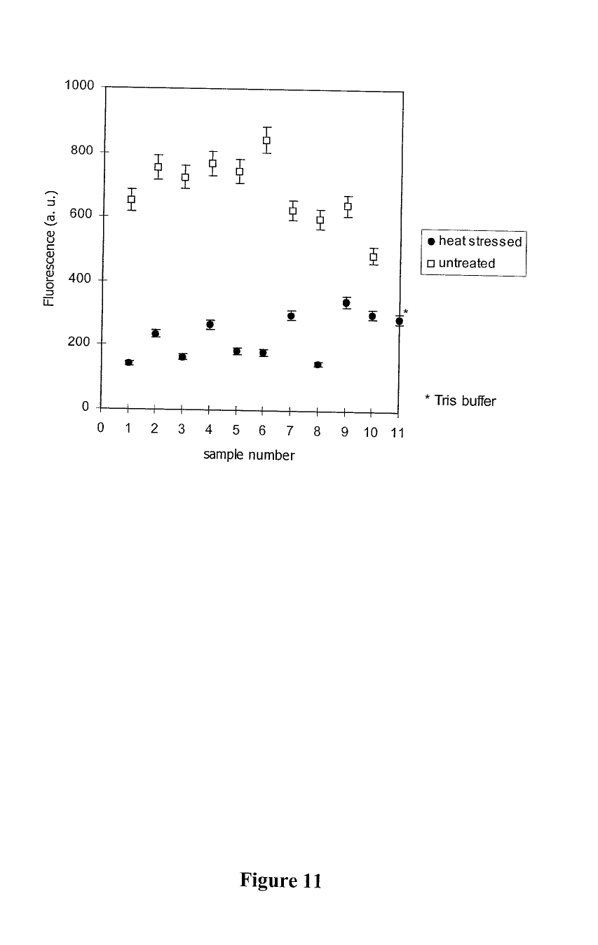

When PicoGreen was used to detect the formation of double stranded DNA in the presence of untreated urine samples, unexpected high fluorescence intensity levels were recorded. Possible explanations for this phenomenon include (1) high concentrations of small single stranded DNA fragments in urine samples can act as random primers for the polymerase enzyme to initial DNA replication, (2) deoxyribonuclease enzymes (DNase) present in the urine sample digest the M13 template into shorter fragments, allowing some of these shorter fragments to anneal to each other to form double stranded DNA, and (3) other proteins may be present which may increase the activity of polymerase enzyme. FIG. 11 demonstrates that after heat stressing urine samples, non-specific fluorescence signals were greatly reduced; however, sample to sample variation still existed. This effect was attributed to the salt concentration effect of urine on the polymerase enzyme activity.

### EXAMPLE 10

### Effect of Urine on DNA Synthesis

The formation of double stranded DNA was also detected by DNA agarose gel electrophoresis. Before samples were subjected to gel electrophoresis, they were purified by Microspin™ G-50 columns. The purified DNA samples were then precipitated with 100% ethanol and redissolved in 50 mM NaOH solution containing 1 mM EDTA to denature the DNA. The DNA was then loaded into the well of the agarose gel, and electrophoresis was conducted. After electrophoresis, the gel was neutralized, stained with ethidium bromide, and photographed under UV light.

To further confirm the presence of DNase or other proteins in urine, denatured DNA gel electrophoresis experiments were conducted. Two samples, #4 and #6 from Example 9, which produced the highest non-specific fluorescence signals were tested by gel electrophoresis. Under denaturing electrophoresis conditions, synthesized double stranded DNA is unwound to form single stranded DNA. The experiment demonstrated that, with or without primer, untreated urine samples caused the fragmentation of the M13 template as was evidenced by the disappearance of M13 bands. The smeared DNA bands below the M13 molecular weight position were low molecular weight fragments of M13. After the urine samples were heat stressed, without the primer, the single stranded M13 band was clearly visible. With primer, the single stranded M13 band was still clearly detectable, with its synthesized complementary single stranded DNA base number ranging from 1 K to 4 K. These results suggested that the urine matrix problem was primarily due to DNase.

These gel electrophoresis experiments also provided information regarding the degree of DNA synthesis. Under normal DNA synthesis conditions, instead of seven thousand bases of dNTP being incorporated, only two thousand bases of dNTP were incorporated per M13 template. Increasing the reaction time did not increase the number of incorporated dNTP. Two reasons may explain the termination of DNA synthesis: (1) the polymerase enzyme denatured during the reaction course, or (2) double stranded DNA in the M13 template (secondary structure) stopped the DNA synthesis.

### EXAMPLE 11

### Inhibition of DNA Synthesis by Biotin Specific Antibody

A universal primer-biotin conjugate was synthesized by Oligos (Wilsonville, Oreg.), free biotin was manufactured by Roche (Nutley, N.J.), and polyclonal antibodies against biotin were obtained from Lampire Biological (Pipersville, Pa.). To test the ability of anti-biotin antibodies on the inhibition of DNA synthesis with the biotin modified universal primer, the following experiments were conducted. In a 96-well microtiter fluorescence plate 5 μl 10 mM Tris buffer, 4×10⁻¹³ moles of biotin modified universal primer in 5 μl of primer buffer were incubated with 5 μl anti-biotin antibodies at various titers for 2 minutes. Antibodies were diluted in 10 mM Tris buffer, pH 7.5. After incubation, other DNA synthesis reagents were added into the well according to the order previously described. The final concentration for primer was 10 nM, for the template 10 nM, for enzyme 2 units per reaction, and for dNTP 0.3 nM. The final buffer concentration was 108 mM, and the reaction time was 10 minutes.

Experiments were also designed to verify the specificity of the antibody-caused inhibition using combinations of modified universal primers and antibodies generated against biotin or morphine or propoxyphene. In a 96-well microtiter fluorescence plate, 5 μl of primer solution containing 4×10⁻¹³ moles of the amino dT C₆ modified universal primer or the biotin modified universal primer were incubated with 10 μl of Tris buffer, pH 7.5, or 10 μl biotin antibodies or 10 μl morphine antibodies or 10 μl of propoxyphene antibodies. All antibodies were diluted in Tris buffer, pH 7.5, at 1:7.5 titer. The mixture was incubated for 10 minutes at room temperature, then other DNA synthesis reagents were added into the well according to the order previously described.

Polyclonal anti-biotin antibodies were used to inhibit the DNA synthesis with the biotin modified universal primer. Once anti-biotin antibody bound to the primer linked biotin, the spatial effect caused by the antibody binding prevented the primer and the template from annealing; or the annealing was allowed, however, the polymerase enzyme could not bind to the primer in an appropriate manner for catalysis. This mechanism of inhibition is yet to be determined. The degree of DNA synthesis inhibition was proportional to the concentration of antibodies.

The inhibition of DNA synthesis by analyte specific antibody (biotin) was specific for the antibody-antigen interaction between biotin and its antibodies, as was evidenced by morphine antibodies or propoxyphene antibodies used at similar total protein concentrations as that of the biotin antibodies could not cause inhibition, and the biotin antibodies could not inhibit the DNA replication with the amino dT C₆ modified primer which was not labeled with biotin.

### EXAMPLE 12

### Detection of Biotin in Buffer

Biotin was dissolved in DMSO at a concentration of 1 mg/ml, then further diluted to various concentrations in Tris buffer, pH 7.5. In each well of a 96-well microtiter fluorescence plate, 5 μl of the diluted biotin solution at various concentrations were incubated with 5 μl of biotin antibody at a selected titer for 2 minutes, then 5 μl of the biotin modified universal primer was added, and the mixture was incubated for another 2 minutes. These incubations were conducted to ensure the interaction between the biotin and its antibody. Other reagents for DNA synthesis were added into each well subsequently, and the reaction time was 10 minutes.

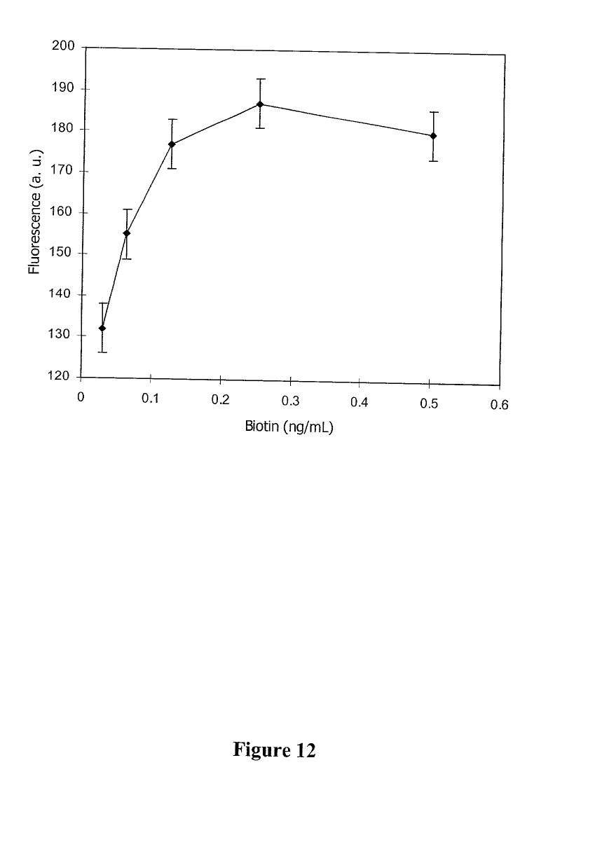

A competition system with free biotin to prevent the antibody inhibition of DNA synthesis with biotin modified universal primer was also investigated. The lowest antibody concentration at which DNA synthesis was completely inhibited with the biotin antibodies was used. Free biotin in the buffer competed with the primer-linked biotin to bind the antibodies, allowing proportional amounts of the primer to anneal to the template, resulting in proportional levels of double stranded DNA. Thus it was demonstrated that a competition binding system can be set up for the detection of biotin and that sensitivities as low as 10⁻¹⁰ M for biotin concentration can be obtained. See FIG. 12.

### EXAMPLE 13

### Carcinoembryonic Antigen (CEA) Binding Assay: Digestion of CEA Antibodies to F(ab′)₂ by Ficin

CEA mouse monoclonal antibodies (RD # 15158-72) and CEA Enzyme Immunoassay Test Kit were produced by Roche Diagnostics Systems (Branchburg, N.J.), ficin, 1-cysteine and N-ethylmaleimide were purchased from Sigma (St. Louis, Mo.), 2-mercaptoethylamine HCl (MEA) and sulfosuccinimidyl 4-(N-maleimidomethyl)-cyclohexane-carboxylate (sulfo-SMCC) were manufactured by Pierce (Rockford, Ill.). SDS-PAGE gel was obtained from BioRad (Hercules, Calif.). Amino dTC₆ modified M13 universal primer was synthesized by Oligos (Wilsonville, Oreg.). The structure of the modified universal primer was 5′ GTA AAA CGX CGG CCA GT 3′ where X is an amino dT C₆ linker.

0.265 ml of 100 mM EDTA solution was added into 13 ml of protein A purified CEA antibodies (4 mg/ml), the mixture was concentrated to 7.5 ml by a Centriprep (Amicon) with a molecular weight cutoff of 50 kDa. Two ml of the concentrated antibodies were diluted with 1.67 ml of 50 mM Tris buffer, pH 7.0, containing 2 mM EDTA. 50 mg of ficin was dissolved in 50 ml of the same Tris buffer to obtain a 1 mg/ml ficin solution. 200 μl of the ficin solution were added to 3.67 ml of the antibody solution containing EDTA. Ficin digestion reaction was activated by adding 100 mM 1-cysteine to the antibodies solution to a final concentration of 1 mM. The reaction was incubated at 37° C. with gentle shaking in aliquots of 500 μl. After four hours of incubation, 39 μl of 100 mM N-ethylmaleimide solution was added into the reaction mixture to stop the digestion. Completion of digestion was verified by SDS-PAGE gel electrophoresis.

### EXAMPLE 14

### Reduction of F(ab′)₂ to Fab′ by MEA

The reduction of F(ab′)₂ to Fab′ was achieved by using a mild disulfide reducing reagent MEA. Experimental procedures followed manufacture's suggestions. Briefly, one ml of 10 mg/ml F(ab′)₂ and Fc mixture (previously digested) in 100 mM sodium phosphate buffer, pH 6.0, containing 5 mM of EDTA, was reacted with MEA with a final concentration of 0.05 M. Incubation took place at 37° C. for 90 minutes. The reaction mixture was allowed to cool to room temperature, and the free MEA was separated from the reduced antibody fragments with a desalting column which was pre-equilibrated with PBS/EDTA buffer.

### EXAMPLE 15

### Preparation of Primer-Fab′ Conjugate

Conjugation between primer and Fab′ was accomplished using a hetero-bifunctional reagent sulfo-SMCC. Experimental procedure for primer-SMCC synthesis was similar to that for Primer-LSD synthesis, except that no organic solvent was used. The desalting column purified antibody fragments were incubated with primer-SMCC overnight at room temperature, the final total antibody protein concentration was 5 mg/ml, for primer-SMCC was 0.18 mg/ml. Reaction buffer was 200 mM sodium phosphate, pH 7.8, containing 25 mM NaCl and 125 mM EDTA. Purification of the primer-Fab′ conjugate was carried out using a HPLC system. Separation of Primer-Fab′ from other reaction components was accomplished using a Protein-Pak DEAE column (Waters) and a mobile phase of 20 mM sodium phosphate with increasing NaCl concentration. Several peak fractions were collected and analyzed. The peak which was eluted at time 24 minutes was identified as the target conjugate.

### EXAMPLE 16

### Antigen Recognition Ability of CEA Antibody Fragments and Primer-Fab′ Conjugate

The CEA recognition ability of intact IgG, F(ab′)₂, Fab′ and primer-Fab′ was examined by CEA EIA kit by Roche. EIA was performed following the manufacture's direction, and a competition assay was set up with the primer-Fab′ and the enzyme labeled antibody in the kit.

### EXAMPLE 17

### DNA Synthesis with F(ab′) Labeled Primer

To test the reactivity of the Fab′ modified universal primer, the amino dT C₆ modified universal primer and the Fab′ modified universal primer were used and compared in DNA synthesis experiments. In each reaction well, the final concentration for both of the primers was 12.6 nM, for the template 6.3 nM, for enzyme 2.5 units per reaction, and for dNTP 0.3 mM. The final buffer concentration was 108 mM, and the incubation was 30 minutes. Other experiment procedures were conducted as described previously.

Results for CEA EIA test indicated that F(ab′)₂, Fab′ and primer-Fab′ can recognize CEA antigen as well as IgG does (FIG. 13). The Fab′ labeled primer was also tested for its ability to direct DNA synthesis. As FIG. 16 indicates, when Fab′ was covalently linked to the 9th position of the universal primer (from 3′ end), polymerase 1 enzyme can still synthesize double stranded DNA.

What is claimed is:

1. A method for determining an analyte in a sample suspected of containing said analyte comprising the steps of:
a. combining said sample with a single stranded DNA template capable of replication and comprising a starting nucleotide sequence, a deoxyribonucleotide precursor, a DNA polymerase enzyme, a receptor capable of binding with said analyte, a compound capable of generating a detectable signal in the presence of double stranded DNA, and a primer, said primer linked to a ligand or analog of said analyte and comprising a sequence complementary to said starting sequence of said template, under conditions favorable for DNA replication,
b. monitoring the generation of double stranded DNA by said enzyme by measuring the signal produced by said compound, and
c. correlating the production of said signal with the presence or amount of said analyte in said sample.

2. The method of claim 1, wherein said analyte is selected from the group consisting of drugs, drug derivatives, hormones, proteins, polypeptides and oligonucleotides.

3. The method of claim 1, wherein said analyte is LSD.

4. The method of claim 1, wherein said primer is amino dT C₆ modified universal primer.

5. The method of claim 1, wherein said template is M13.

6. The method of claim 1, wherein said receptor is selected from the group consisting of antibodies, antibody fragments and antibody derivatives.

7. The method of claim 1, wherein said compound capable of generating a detectable signal is a DNA intercalator selected from the group consisting of PicoGreen, acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine and YOYO-1.

8. The method of claim 1, wherein said intercalator is PicoGreen.

9. A reagent for determining an analyte in a sample via a method involving inhibition of DNA replication, said reagent comprising a primer bound to a ligand of said analyte or an analog of said analyte.

10. A method for determining an analyte in a sample suspected of containing said analyte comprising the steps of:
a. combining said sample with a single stranded DNA template capable of replication and comprising a starting nucleotide sequence, a deoxyribonucleotide precursor, a DNA polymerase enzyme, said enzyme linked to a ligand or analog of said analyte, a receptor capable of binding with said analyte, a compound capable of generating a detectable signal in the presence of double stranded DNA, and a primer, said primer comprising a sequence complementary to said starting sequence of said template, under conditions favorable for DNA replication,
b. monitoring the generation of double stranded DNA by said enzyme by measuring the signal produced by said compound, and
c. correlating the production of said signal with the presence or amount of said analyte in said sample.

11. The method of claim 10, wherein said analyte is selected from the group consisting of drugs, drug derivatives, hormones, proteins, polypeptides and oligonucleotides.

12. The method of claim 10, wherein said primer is amino dT C₆ modified universal primer.

13. The method of claim 10, wherein said template is M13.

14. The method of claim 10, wherein said receptor is selected from the group consisting of antibodies, antibody fragments and antibody derivatives.

15. The method of claim 10, wherein said compound capable of generating a detectable signal is a DNA intercalator selected from the group consisting of PicoGreen, acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine and YOYO-1.

16. The method of claim 10, wherein said intercalator is PicoGreen.

17. A reagent for determining an analyte in a sample via a method involving inhibition of DNA replication, said reagent comprising a DNA polymerase enzyme linked to a ligand or analog of said analyte.

18. A method for determining an analyte in a sample suspected of containing said analyte comprising the steps of:
a. combining said sample with a single stranded DNA template capable of replication and comprising a starting nucleotide sequence, a deoxyribonucleotide precursor, a DNA polymerase enzyme, a compound capable of generating a detectable signal in the presence of double stranded DNA, and a primer, said primer linked to a receptor capable of binding with said analyte and comprising a sequence complementary to said starting sequence of said template, under conditions favorable for DNA replication,
b. monitoring the generation of double stranded DNA by said enzyme by measuring the signal produced by said compound, and
c. correlating the production of said signal with the presence or amount of said analyte in said sample.

19. The method of claim 18, wherein said analyte is selected from the group consisting of drugs, drug derivatives, hormones, proteins, polypeptides and oligonucleotides.

20. The method of claim 18, wherein said analyte is carcinoembryonic antigen.

21. The method of claim 18, wherein said primer is amino dT C₆ modified universal primer.

22. The method of claim 18, wherein said template is M13.

23. The method of claim 18, wherein said receptor is selected from the group consisting of antibodies, antibody fragments and antibody derivatives.

24. The method of claim 18, wherein said compound capable of generating a detectable signal is a DNA intercalator selected from the group consisting of PicoGreen, acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine and YOYO-1.

25. The method of claim 18, wherein said intercalator is PicoGreen.

26. A reagent for determining an analyte in a sample via a method involving inhibition of DNA replication, said reagent comprising a primer bound to a receptor capable of binding with said analyte

27. A test kit for the determination of an analyte comprising in packaged combination:
a single stranded DNA template capable of replication and comprising a starting nucleotide sequence,
a primer linked to a ligand or analog of said analyte, said primer comprising a sequence complementary to said starting sequence of said template,
a DNA polymerase enzyme,
a deoxyribonucleotide precursor,
a receptor capable of binding with said analyte, and
a compound capable of generating a detectable signal in the presence of double stranded DNA.

28. The kit of claim 27, wherein said analyte is selected from the group consisting of drugs, drug derivatives, hormones, proteins, polypeptides and oligonucleotides.

29. The kit of claim 27, wherein said analyte is LSD.

30. The kit of claim 27, wherein said primer is amino dT C₆ modified universal primer.

31. The kit of claim 27, wherein said template is M13.

32. The kit of claim 27, wherein said receptor is selected from the group consisting of antibodies, antibody fragments and antibody derivatives.

33. The kit of claim 27, wherein said compound capable of generating a detectable signal is a DNA intercalator selected from the group consisting of PicoGreen, acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine and YOYO-1.

34. The kit of claim 27, wherein said intercalator is PicoGreen.

35. A test kit for the determination of an analyte comprising in packaged combination:
a single stranded DNA template capable of replication and comprising a starting nucleotide sequence,
a primer linked to a receptor capable of binding with said analyte, said primer comprising a sequence complementary to said starting sequence of said template,
a DNA polymerase enzyme,
a deoxyribonucleotide precursor, and
a compound capable of generating a detectable signal in the presence of double stranded DNA.

36. The kit of claim 35, wherein said analyte is selected from the group consisting of drugs, drug derivatives, hormones, proteins, polypeptides and oligonucleotides.

37. The kit of claim 35, wherein said analyte is carcinoembryonic antigen.

38. The kit of claim 35, wherein said primer is amino dT C₆ modified universal primer.

39. The kit of claim 35, wherein said template is M13.

40. The kit of claim 35, wherein said receptor is selected from the group consisting of antibodies, antibody fragments and antibody derivatives.

41. The kit of claim 35, wherein said compound capable of generating a detectable signal is a DNA intercalator selected from the group consisting of PicoGreen, acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine and YOYO-1.

42. The kit of claim 35, wherein said intercalator is PicoGreen.

43. A test kit for the determination of an analyte comprising in packaged combination:
a single stranded DNA template capable of replication and comprising a starting nucleotide sequence,
a primer comprising a sequence complementary to said starting sequence of said template,
a DNA polymerase enzyme, said enzyme linked to a ligand or analog of said analyte,
a deoxyribonucleotide precursor, and
a compound capable of generating a detectable signal in the presence of double stranded DNA.

44. The kit of claim 43, wherein said analyte is selected from the group consisting of drugs, drug derivatives, hormones, proteins, polypeptides and oligonucleotides.

45. The kit of claim 43, wherein said primer is amino dT C₆ modified universal primer.

46. The kit of claim 43, wherein said template is M13.

47. The kit of claim 43, wherein said receptor is selected from the group consisting of antibodies, antibody fragments and antibody derivatives.

48. The kit of claim 43, wherein said compound capable of generating a detectable signal is a DNA intercalator selected from the group consisting of PicoGreen, acridine orange, ethidium monoazide, ethidium bromide, propidium iodide, 7-aminoactinomycin D, LDS-751, ACMA, DAPI, dihydroethidium, ethidium homodimers, FluoroNissl Green, hexidium iodide, bisbenzimide, hydroxystilbamidine and YOYO-1.

49. The kit of claim 43, wherein said intercalator is PicoGreen.
